(19) United States
(12) Patent Application Publication
(10) Pub. No.: US 2014/0080908 A1
(43) Pub. Date: Mar. 20, 2014

(54) COMPOSITIONS AND METHODS FOR IMPROVING OR PREVENTING DEPRESSION-LIKE SYMPTOMS

(71) Applicant: PHYTO TECH CORP., Rancho Santa Margarita, CA (US)

(72) Inventor: Steven CHEN, Rancho Santa Margarita, CA (US)

(73) Assignee: PHYTO TECH CORP., Rancho Santa Margarita, CA (US)

(21) Appl. No.: 14/032,134

(22) Filed: Sep. 19, 2013

Related U.S. Application Data
(60) Provisional application No. 61/703,227, filed on Sep. 19, 2012.

(51) Int. Cl. A61K 31/191

(52) U.S. Cl. CPC A61K 31/191
USPC 514/557

(57) ABSTRACT

The invention provides methods of improving and/or preventing one or more depression-like symptoms by administering to an individual an effective amount of calcium L-threonate. Also provided are related kits and articles of manufacture.

### CROSS-REFERENCE TO RELATED APPLICATIONS

This application claims the benefit of U.S. Provisional Patent Application No. 61/703,227, filed Sep. 19, 2012, the disclosure of which is hereby incorporated by reference in its entirety.

### FIELD OF THE INVENTION

This invention relates to compositions and methods to improve or prevent depression-like symptoms by the administration of an effective amount of calcium L-threonate.

### BACKGROUND OF THE INVENTION

Calcium (Ca²⁺) is a chemical element that is critical for our physical and mental health. About 99% of the body's Ca²⁺ is stored in the bones and teeth, and the remaining Ca²⁺ plays roles in a variety of physical functions, including muscle contraction and electrical conduction in heart muscle. Ca²⁺ also plays a role in the release of neurotransmitters, i.e., chemicals that serve as messengers between cells within the nervous system and in the regulation of cellular signaling pathways. Severe Ca²⁺ deficiencies are known to cause a variety of physical disorders, such as rickets, poor blood clotting, and osteoporosis. Ca²⁺ deficiencies are thought to contribute to mood and mental health disorders, as well. For example, short term, mild deficiency of Ca²⁺ is thought to cause nerve sensitivity, twitching muscles, brittle nails, palpitations, and mood and behavior disturbances including dysphoria and insomnia. More severe deficiencies are thought to cause muscle cramps, numbness, stiffness of hands, abnormal heartbeat, tingling of extremities, and depression. However, it was not clear that there is a relationship between Ca²⁺ and depression (Bertone-Johnson et al. (2012) “Vitamin D Supplementation and Depression in the Women's Health Initiative Calcium and Vitamin D Trial.” Am J Epidemiol 176: 1-13; Chakroborty et al. (2012) “Early presynaptic and postsynaptic calcium signaling abnormalities mask underlying synaptic depression in presymptomatic Alzheimer's disease mice.” J Neurosci 32: 8341-8353; Derom et al. (2012) “Magnesium intake is not related to depression risk in Spanish university graduates.” J Nutr 142: 1053-1059; Jamilian et al. (2012) “Vitamin D, parathyroid hormone, serum calcium and phosphorus in patients with schizophrenia and major depression.” Int J Psychiatry Clin Pract. Apr 26).

Nutritional supplements provide calcium in a variety of forms, e.g., calcium carbonate, calcium acetate, calcium citrate, calcium gluconate, etc. The different forms of calcium exhibit different dissolution characteristics which affect absorption of Ca²⁺ from the intestine. One form of calcium, calcium L-threonate, has been shown to exhibit a higher bioavailability as compared to calcium gluconate, calcium acetate, and calcium carbonate (Aguilar et al. (2008) “Scientific Opinion of the Panel on Food Additives and Nutrient Sources added to food (ANS) on a request from the Commission on calcium L-threonate as a source for calcium added for nutritional purposes in food supplements.” The EFSA Journal 866: 1-20). Calcium L-threonate is currently used to treat or prevent a variety of bone- and cartilage-related disorders, including bone fracture, osteoporosis, rickets, osteoarthritis, rheumatoid arthritis, multiple chondritis, articular cartilage damage, and osteochondrosis (see, e.g., U.S. Pat. No. 6,077,872; U.S. Pat. No. 6,713,513; U.S. Pat. No. 6,727,288; WO 2003/004012; and WO 2003/004011).

Depression is a common mental disorder that is estimated to affect approximately 121 million people worldwide. Depression causes persistent feelings of sadness and loss of interest. Accordingly, depression can lead to a variety of emotional and physical problems that significantly impair in an individual's ability to take care of his or her daily responsibilities. At its worst, depression can lead to suicide, a tragic fatality associated with the loss of approximately 850,000 lives every year.

The data in the present application indicated that calcium L-threonate could significantly improve or prevent one or more depression-like symptoms. Therefore, it is an object of the present invention to provide a method for improving or preventing depression-like symptoms in an individual. It is another object of the present invention to provide a composition comprising calcium L-threonate for improving or preventing depression-like symptoms in an individual. It is another object of the present invention to provide calcium L-threonate in the preparation of a composition for improving or preventing depression-like symptoms in an individual.

All references cited herein, including patent applications and publications, are hereby incorporated by reference in their entireties.

### BRIEF SUMMARY OF THE INVENTION

In one aspect, the invention provides a method of improving or preventing depression-like symptoms comprising administering an effective amount of calcium L-threonate to an individual.

In some embodiments, the calcium L-threonate is administered to a human individual at a dosage of about 500 mg/day to about 1500 mg/day. In some embodiments, the calcium L-threonate is administered to a human individual at a dosage of about 600 mg/day to about 1200 mg/day. In some embodiments, the calcium L-threonate is administered to the individual at a dosage of about 8.57 mg/kg/day to about 21.43 mg/kg/day. In some embodiments, the calcium L-threonate is administered for at least 3 months, at least 6 months, or at least 9 months.

In some embodiments, the calcium L-threonate is administered orally. For example, in some embodiments, calcium L-threonate is provided as a nutritional supplement. In some embodiments, calcium L-threonate is provided in a composition comprising calcium L-threonate and a pharmaceutically acceptable carrier.

In some embodiments, the individual is an adult, e.g., a human individual who is 18 years of age or older. In some embodiments, the individual is a child, e.g., a human individual who is less than 18 years of age. In some embodiments, the individual has been diagnosed with depression. In some embodiments, the individual has been selected for administration of calcium L-threonate based on having depression or depression-like symptoms. In some embodiments, the individual has anxiety and/or chronic stress.

In some embodiments, the administration of calcium L-threonate improves one or more of the depression-like symptoms in the individual. In some embodiments, the symptom that is improved is a despair-based symptom or an anxiety-based symptom.

In another aspect, the invention provides calcium L-threonate for use in improving and/or preventing depression-like symptoms. Also provided herein is the use of calcium L-threonate in the manufacture of a medicament. In some embodiments, the medicament is for improving and/or preventing depression-like symptoms, including despair-based and/or anxiety-based symptoms.

In another aspect, the invention provides kits or articles of manufacture comprising (a) a composition comprising calcium L-threonate, and (b) a package insert or a label indicating that composition may be useful for improving or preventing one or more depression-like symptoms. In some embodiments, the kits or articles of manufacture comprise a nutritional supplement comprising calcium L-threonate, e.g., in a tablet or a capsule, packaged in a container with an insert or label indicating that the product is useful for improving and/or preventing depression-like symptoms. In some embodiments, the insert or label may indicate that the composition has a stress-reducing and/or anxiety-reducing effect. In some embodiments, the insert or label indicates the ingredients of the composition comprise calcium L-threonate. In some embodiments, the package insert or the label is in a position which is visible to prospective purchasers.

It is to be understood that one, some, or all of the properties of the various embodiments described herein may be combined to form other embodiments of the present invention. These and other aspects of the invention will become apparent to one of skill in the art.

### BRIEF DESCRIPTION OF THE DRAWINGS

FIG. 1 provides the results of experiments performed to compare the absorption of calcium L-threonate and calcium gluconate by human bone marrow cells at various time points.

FIG. 2 provides the results of experiments performed to determine the effect of calcium L-threonate administration on despair-based behavior in mice. FIG. 2A shows the duration of immobility in TST (tail suspension test) for the control group (vehicle), calcium gluconate treated group (Ca-gluconate), and calcium L-threonate treated group (Ca-L-threonate). FIG. 2B shows the duration of immobility in FST (forced-swimming test) for the control group (vehicle), calcium gluconate treated group (Ca-gluconate), and calcium L-threonate treated group (Ca-L-threonate).

FIG. 3 provides the results of experiments performed to determine the effect of calcium L-threonate on anxiety-based behavior in mice.

### DETAILED DESCRIPTION OF THE INVENTION

The invention provides, inter alia, methods of improving or preventing depression-like symptoms by administering an effective amount of calcium L-threonate to an individual. The methods of the invention are based on the unexpected observation that administering calcium L-threonate improves or prevents depression-like symptoms. For example, the methods provided by the invention benefit adults or children suffering from anxiety-based symptoms (such as excessive worrying, feelings of anxiousness, apprehension, unease, fear, and/or dread), and/or despair-based symptoms (such as feelings of hopelessness, helplessness, powerlessness, pessimism and/or anguish).

### A. Definitions

As used herein, “depression-like symptoms” refer to a combination of symptoms (described below) that disrupt or limit an individual's ability to, e.g., perform typical daily activities or enjoy pleasurable activities, for a period of at least two weeks. Psychological symptoms include, but are not limited to, e.g., difficulty concentrating; indecisiveness; feelings of guilt, worthlessness and/or helplessness; feelings of hopelessness and/or pessimism; irritability; restlessness or lack of energy; anhedonia; anger; increased drug or alcohol abuse; persistent feelings of sadness, “emptiness,” or despair; anxiety; becoming withdrawn or isolated; neglect of personal hygiene; and/or thoughts of suicide. The physical symptoms include, but are not limited to, e.g., fatigue and/or decreased energy; insomnia, early-morning wakefulness, or excessive sleeping; overeating or appetite loss; and/or persistent aches, pains, cramps, or digestive problems. The diagnosis of depression is based on an individual's self-reported experiences, behavior reported by relatives or friends, and a mental status examination by a psychiatrist. Additional diagnostic criteria for depression are detailed in the current version of the Diagnostic and Statistical Manual of Mental Disorders (i.e., DSM-IV TR).

As used herein, “improving depression-like symptoms” refers to the ability to reduce or reverse the number of depression-like symptoms and/or the intensity of one or more depression-like symptoms.

As used herein “preventing depression-like symptoms” refers to the ability to keep an individual from experiencing one or more depression-like symptoms or experiencing an increase in the intensity of one or more depression-like symptoms, as described herein.

As used herein, an “effective amount” refers to at least an amount effective, at dosages and for periods of time necessary, to achieve the desired result, e.g., a reduction in the number of depression-like symptoms or in the intensity of one or more depression-like symptoms, as described elsewhere herein, or, e.g., the prevention of one or more depression-like symptoms or of the intensity of one or more depression-like symptoms, as described elsewhere herein. An effective amount can be provided in one or more administrations.

An “individual” or a “subject” is a mammal, more preferably a human. Mammals also include, but are not limited to, farm animals, sport animals, pets (such as cats, dogs, horses), primates, mice and rats.

As used herein, the term “adult” refers to a human individual that is at least 18 years old, whereas the term “child” refers to a human individual that is less than 18 years old.

As used herein, “anxiety” refers to excessive, long-lasting angst or worry about events, objects, and/or situations. Symptoms of anxiety include, but are not limited to, e.g., excessive worrying, feelings of anxiousness, apprehension, unease, fear, and/or dread; avoidance and/or shyness; increased heart rate, heart palpitations, and/or increased blood pressure, digestive distress (e.g., nausea, stomach pain, vomiting, and/or diarrhea); panic attacks; tingling and/or numbness in the hands and/or feet; chest pain, sweating, weakness, dry mouth, and/or shortness of breath; sleep problems (e.g., disrupted sleep, nightmares, and/or insomnia); expectations of failure and/or disaster; and/or uncontrollable, obsessive thoughts of unpleasant or traumatic experiences.

As used herein, “chronic stress” refers to one or more of the physical, psychological, and/or emotional responses that an individual exhibits during a prolonged period of distressing events over which the individual perceives he or she has no control. The symptoms of chronic stress, include, but are not limited to, e.g., gingivitis, upset stomach or other digestive discomfort, dizziness and/or backache, insomnia, chest discomfort, frustration, anger, moodiness, anxiousness, nervousness, aggression, intense mood swings, rash, poor concentration, heightened confusion in stressful situations, inability to complete tasks, unstable blood pressure, hemorrhoids, varicose veins, panic attacks, and suicidal thoughts.

As used herein, the singular form “a”, “an”, and “the” includes plural references unless indicated otherwise.

Reference to “about” a value or parameter herein refers to the usual error range for the respective value readily known to the skilled person in this technical field. Reference to “about” a value or parameter herein includes (and describes) aspects that are directed to that value or parameter per se. For example, description referring to “about X” includes description of “X.”

It is understood that aspects and embodiments of the invention described herein include “comprising,” “consisting,” and “consisting essentially of” aspects and embodiments.

### B. Methods of Improving or Preventing Depression-Like Symptoms

In one aspect, the invention provides methods for improving or preventing depression-like symptoms comprising administering an effective amount of calcium L-threonate to an individual.

In another aspect, the invention provides method for treating or preventing depression comprising administering an effective amount of calcium L-threonate to an individual. As used herein, the “treatment” or “treating” is an approach for obtaining beneficial or desired results including clinical results. For purposes of this invention, beneficial or desired clinical results include, but are not limited to, one or more of the following: decreasing symptoms resulting from the disease, increasing the quality of life of those suffering from the disease, decreasing the dose of other medications required to treat the disease and/or delaying the progression of the disease. As such, it is not required that all effects of the condition be entirely reversed or the progression of the diseases be completely halted.

In certain embodiments of the methods, the individual has depression, depression-like symptoms, or has been diagnosed with depression. In certain embodiments, the individual has been selected for treatment or administration based on having depression, having depression-like symptoms, or having been diagnosed with depression. In certain embodiments, the individual is a human.

In certain embodiments of the methods, calcium L-threonate is administered at a dosage of about 400 mg/day to about 2500 mg/day for a human. For example, calcium L-threonate is administered at a dosage of at least about 400 mg/day, at least about 500 mg/day, at least about 600 mg/day, at least about 700 mg/day, at least about 800 mg/day, at least about 900 mg/day, at least about 1000 mg/day, at least about 1100 mg/day, at least about 1200 mg/day, at least about 1300 mg/day, at least about at least about 1400 mg/day, at least about 1500 mg/day, at least about 1600 mg/day, at least about 1700 mg/day, at least about 1800 mg/day, at least about 1900 mg/day, at least about 2000 mg/day, at least about 2100 mg/day, at least about 2200 mg/day, at least about 2300 mg/day, at least about 2400 mg/day, less than about 2500 mg/day, or about 2500 mg/day, including any range in between these values.

In certain embodiments, calcium L-threonate is administered at a dosage of about 600 mg/day to 1200 mg/day, and no more than 1500 mg/day, for a human. For example, calcium L-threonate is administered at a dosage of at least about 600 mg/day, at least about 650 mg/day, at least about 700 mg/day, at least about 750 mg/day, at least about 800 mg/day, at least about 850 mg/day, at least about 900 mg/day, at least about 950 mg/day, at least about 1000 mg/day, at least about 1050 mg/day at least about 1100 mg/day, at least about 1150 mg/day, at least about 1200 mg/day, or more than 1200 mg/day, e.g., at least about 1250 mg/day, at least about 1300 mg/day, at least about at least about 1350 mg/day, at least about 1400 mg/day, at least about 1450 mg/day, less than about 1500 mg/day or about 1500 mg/day, including any range in between these values.

In certain embodiments, calcium L-threonate is administered at a dosage of about 8.57 mg/kg/day to about 21.43 mg/kg/day. For example, L-threonate is administered at a dosage of at least about 8.57 mg/kg/day, at least about 9.64 mg/kg/day, at least about 10.71 mg/kg/day; at least about 11.78 mg/kg/day; at least about 12.85 mg/kg/day; at least about 13.92 mg/kg/day; at least about 14.99 mg/kg/day; at least about 16.06 mg/kg/day; at least about 17.13 mg/kg/day; at least about 18.2 mg/kg/day; at least about 19.27 mg/kg/day; at least about 20.34 mg/kg/day; less than about 21.43 mg/kg/day, or more than about 21.43 mg/kg/day, including any range in between these values. For example, the calcium L-threonate is administered at a dosage of at least about 23.4 mg/kg/day, at least about 25.37 mg/kg/day, at least about 27.34 mg/kg/day, at least about 29.31 mg/kg/day, at least about 31.28 mg/kg/day, at least about 33.25 mg/kg/day, at least about 35.22 mg/kg/day, at least about 37.19 mg/kg/day, at least about 39.16 mg/kg/day, at least about 41.13 mg/kg/day, at least about 43.1 mg/kg/day, less than about 45 mg/kg/day, or about 45 mg/kg/day.

In certain embodiments, the calcium L-threonate is administered to the individual for at least about 1 month, for at least about 1.5 months, for at least about 2 months, for at least about 2.5 months, for at least about 3 months, or for more than three months. For example, calcium L-threonate is administered for at least about 3.5 months, for at least about 4 months, for at least 4.5 months, for at least 5 months, for at least 5.5 months, for at least about 6 months, for at least about 6.5 months, for at least about 7 months, for at least about 7.5 months, for at least about 8 months, for at least about 8.5 months, for at least about 9 months, or for more than 9 months, e.g., for at least about 10 months, for at least about 11 months, for at least 11.5 months, for at least about 12 months, for at least about 18 months, for at least about 24 months, or for more than 24 months, including any range in between these values.

In certain embodiments, the administration of calcium L-threonate improves one or more depression-like symptoms in the individual. In certain embodiments, one or more of the depression-like symptoms are improved after administration of calcium L-threonate for at least about one month, at least about two months, at least about three months, or at least about four months, including any range in between these values. In certain embodiments, the calcium L-threonate is administered daily.

The calcium L-threonate is administered using any method well known in the art, e.g., via ingestion, inhalation, injection, or mucosal administration. In certain embodiments, the calcium L-threonate is administered orally. In certain embodiments, the calcium L-threonate is provided in a solid dosage forms, such as capsules, tablets, and powders, or in liquid dosage forms, such as elixirs, syrups, and suspensions. In certain embodiments, the calcium L-threonate is provided as a nutritional supplement, e.g., a calcium L-threonine nutritional supplement available from BioCalth® (e.g., BioCalth® Caplets, BioCalth® Chewables, BioBlast® Men, BioBlast® Women, BioCalth® Kids, etc.). In certain embodiments, the calcium L-threonate is administered in a composition comprising calcium L-threonate and a pharmaceutically acceptable carrier, e.g., a carrier, excipient, or stabilizer that is nontoxic to the individual being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

In certain embodiments of the methods, the individual to whom the calcium L-threonate is administered is a human. In certain embodiments, the individual is an adult, e.g., an individual that is 18 years old or older. In certain embodiments of the methods, the individual is a child, i.e., an individual that is less than 18 years old. In certain embodiments of the methods, the individual has been diagnosed with depression or having one or more of depression-like symptoms described below: difficulty concentrating; indecisiveness; feelings of guilt, worthlessness and/or helplessness; feelings of hopelessness and/or pessimism; irritability; restlessness or lack of energy; anhedonia; anger; increased drug or alcohol abuse; persistent feelings of sadness, “emptiness,” or despair; anxiety; becoming withdrawn or isolated; neglect of personal hygiene; thoughts of suicide; fatigue and/or decreased energy; insomnia, early-morning wakefulness, or excessive sleeping; overeating or appetite loss; and/or persistent aches, pains, cramps, or digestive problems. In certain embodiments, the diagnosis of depression is based on the experiences an individual reports to a physician or psychologist, the behavior reported by relatives or friends to a physician or psychologist, and/or a mental status examination by a psychiatrist. In certain embodiments of the methods, the individual has been selected for administration of calcium L-threonate based on having depression, having depression-like symptoms, or being diagnosed with depression. In certain embodiments, the individual also suffers from anxiety and/or chronic stress, as described elsewhere herein.

In certain embodiments, the administration of calcium L-threonate improves one or more of the depression-like symptoms, e.g., symptoms described elsewhere herein. In certain embodiments the symptom that is improved is an anxiety-based symptom, including, but not limited to, e.g., excessive worrying, feelings of anxiousness, apprehension, unease, or dread; avoidance and/or shyness; increased heart rate, heart palpitations, and/or increased blood pressure; panic attacks; digestive distress (e.g., nausea, stomach paint, vomiting, and/or diarrhea); tingling and/or numbness in the hands and/or feet; chest pain, sweating, weakness, dry mouth, and/or shortness of breath; sleep problems (i.e., disrupted sleep, nightmares, and/or insomnia); and/or uncontrollable, obsessive thoughts of unpleasant or traumatic experiences. In certain embodiments, the symptom that is improved is a despair-based symptom, including, but not limited to, e.g., hyperventilation, loss of appetite, insomnia, mood swings, irritability, lack of concentration, feelings of hopelessness, helplessness, powerlessness, pessimism and/or anguish; feeling overwhelmed; and/or thoughts of suicide.

Calcium L-Threonate

Calcium L-threonate is calcium (2R, 3S)-2,3,4-trihydroxybutyric acid with the molecular formula C₈H₁₄O₁₀Ca and structural formula:

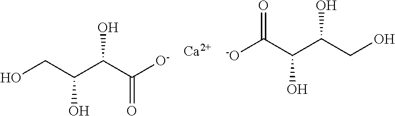

The molecular weight of calcium L-threonate is 310.3 g/mol, and its CAS (Chemical Abstracts Service) Number is 70753-61-6. This compound can be produced by hydrogen peroxide oxidation of the calcium salt, obtained from ascorbic acid and calcium carbonate. Additional methods of producing calcium L-threonate are known in the art.

For example, the compound can be prepared by the following steps: a certain amount of L-ascorbic acid is added to water and dissolved, then calcium carbonate is slowly added into the mixture with stirring. To the above mixture, hydrogen peroxide is added dropwise at a temperature between 10° C. and 60° C. and the temperature is maintained for 1-4 hours at 40-80° C. After active charcoal is added, the mixture is filtered. The filtrate is concentrated at a temperature between 30° C. and 90° C. and crystallized at ambient temperature. The crystal is dried at a temperature of 50-100° C. In the above process of preparing calcium L-threonate, the addition of calcium carbonate is carried out very slowly to avoid loss of material out of the container due to production of carbon dioxide gas. The above process of preparing calcium L-threonate may further comprise the operation of washing the cake obtained by filtering the mixture that has been treated with active charcoal, twice with hot water of 80° C. and the operation of concentrating the combined washes and filtrate. Methods for producing calcium L-threonate are described in U.S. Pat. No. 6,077,872; U.S. Pat. No. 6,713,513; U.S. Pat. No. 6,727,288; WO 2003/004012; and WO 2003/004011, the contents of which are hereby incorporated by reference in their entireties.

Typically, calcium L-threonate is administered orally in various forms of formulations such as tablets and capsules, i.e., made according to methods known in the art, and other forms of pharmaceutically acceptable compositions. For example, calcium L-threonate is provided pharmaceutical composition comprising calcium L-threonate and a pharmaceutically acceptable carrier, e.g., any one of a variety of carriers that have been widely employed in medicaments in the prior art such as excipients. Pharmaceutical compositions comprising calcium L-threonate may also contain other optional ingredients, e.g., perfumes, colorants, flavoring agents, and/or sweeteners. A tablet, capsule, or pharmaceutical composition comprising calcium L-threonate that is used in the methods of the present invention contains at least 60%, at least about 70%, at least about 80%, at least about 90%, at least about 91%, at least about 92%, at least about 93%, at least about 94%, at least about 95%, or more than 95%, e.g., at least about 96%, at least about 97%, or at least about 98% of calcium L-threonate, including ranges between these values.

Dosages of calcium L-threonate can also be provided as nutritional supplements to be taken with meals or as food additives that can be mixed into drinks, added to condiments, or added to solid food during or following its preparation.

Calcium L-threonate can also be formulated for parenteral administration, inhalation, or mucosal administration using methods well known to those of skill in the art. Methods of preparing such formulations are described in Remington's Pharmaceutical Sciences, 18th edition, A. Gennaro, ed., Mack Publishing Co., Easton, Pa., 1990; and Remington, The Science and Practice of Pharmacy 20th Ed. Mack Publishing, 2000.

The powder form of calcium L-threonate is commercially available from a variety of manufacturers, including, but not limited to, e.g., Shaanxi Tianrun Phytochemical Co., Ltd., Rm. 806 Palmary Plaza No. 4, Fenghui Nan Road, High-Tech Zone, Xi'an Shaanxi, China; Shanghai Soyoung Biotechnology, Inc., 40-601, Lane 688, Fanjin Road, Pudong New Area, Shanghai, China; Ningbo Jiangdong Healtrue Biotechnology Co., Ltd., No. 25 Lane 280 Xingning Road, Zhejiang, Ningbo, China; and Bio-Sugars Technology Co., Ltd., Majian, Lanxi, Zhejiang, China. Calcium L-threonate capsules and tablets are commercially available and are distributed by BioCalth International Corp., 1871 Wright Avenue, La Verne, Calif. 91750, USA.

### C. Kits and Articles of Manufacture

The invention also provides kits and articles of manufacture comprising a composition comprising calcium L-threonate, e.g., packaged in a container. The container may comprise an insert or a label indicating that the composition is useful for improving or preventing one or more depression-like symptoms, including anxiety- or despair-based symptoms, and/or treating or preventing depression. In certain embodiments, the kits or articles of manufacture comprise a nutritional supplement comprising calcium L-threonate (for example, in a tablet or capsule), packaged in a container with an insert or a label indicating that the product is useful for improving or preventing one or more depression-like symptoms, including anxiety- or despair-based symptoms. In certain embodiments, the insert or label may further indicate that the composition or product has an anxiety-reducing and/or despair-reducing effect. In certain embodiments, the insert or label indicates the ingredients of the composition inside include calcium L-threonate.

### EXAMPLES

The following examples are offered to illustrate, but not to limit the claimed invention. It is understood that the examples and embodiments described herein are for illustrative purposes only and that various modifications or changes in light thereof will be suggested to persons skilled in the art and are to be included within the spirit and purview of this application and scope of the appended claims.

### Example 1

### Use of Calcium L-Threonate in the Improvement and/or Prevention of Depression-Like Symptoms

A high calcium intake has been associated with many health benefits, including a lower risk for kidney stones (Curhan et al. (1993) “A prospective study of dietary calcium and other nutrients and the risk of symptomatic kidney stones.” The New England Journal of Medicine 328: 833-838; Bihl et al. (2001) “Recurrent renal stone disease-advances in pathogenesis and clinical management.” Lancet 358: 651-656; Hall et al. (2001) “Risk factors for kidney stones in older women in the Southern United States.” Am J Med Sci 322: 12-18). The recommended dietary allowance (RDA) for calcium varies based on an individual's age, sex, and general health. It is well known that adults 19-50 years need 1000 mg/day and adult women 51-70 years need 1,200 mg/day. However, numerous factors, including, e.g., age, vitamin D intake, and diet, affect calcium absorption. Calcium absorption also varies depending on the source of calcium, how much calcium is consumed at one time, and how the calcium it is formulated. Therefore, calcium formulations that are more efficiently absorbed are desirable.

There are more than 100 commercially available calcium-containing nutritional supplements. For example, many supplements provide calcium in the form of calcium gluconate, which is formed by the neutralization of gluconic acid with lime or calcium carbonate. Calcium L-threonate, which is also available as a nutritional supplement, contains calcium (Ca²⁺) and L-threonate (C₈H₁₄O₁₀Ca), a bioactive metabolite of vitamin C in vivo. L-threonate is a Ca²⁺ biocarrier.

To determine whether calcium L-threonate can be absorbed significantly by human cells, human bone marrow (obtained from surgery procedures) was cultured in MEM containing 10% fetal bovine serum (FBS), 2.5 g/ml Fungizone® and 50 g/ml gentamicin (standard medium) until near confluence (approximately, 10-15 days). At this stage, adherent cells were enzymatically released (0.04% trypsin and 0.025% collagenase), counted using a hemocytometer, and cultured at a density of 10⁴ cells cm⁻², in 96-well culture plates, for periods of up to 42 days. Separate cultures were grown under the following experimental conditions: (1) standard medium; (2) standard medium+AA (ascorbic acid) (50 μg/ml); (3) standard medium + GP (β-glycerophosphate) (10 mmol); (4) standard medium+Dex (dexamethasone) (10 nmol); (5) standard medium+AA+GP; and (6) standard medium+AA+GP+Dex.

The cell morphology, cell growth parameters (cell viability/proliferation and total protein content) and functional activity (alkaline phosphatase activity and ability to form calcium phosphate deposits) of each culture was characterized using standard procedures known in the art at days 3, 7, 14, 21, 28, 35 and 42. Cultures were incubated at 37° C. in a humidified atmosphere of 95% air and 5% CO₂, and culture medium was changed twice a week; monitoring of the cultures was done daily using phase contrast inverted microscopy. Bone marrow cells cultured in the six experimental conditions described were observed during the initial phase of the culture, i.e., at 30 minutes, 1 hour, 4 hours and 24 hours after being plated, and also throughout the culture period. Cells were observed by phase contrast microscopy and scanning electron confocal microscopy.

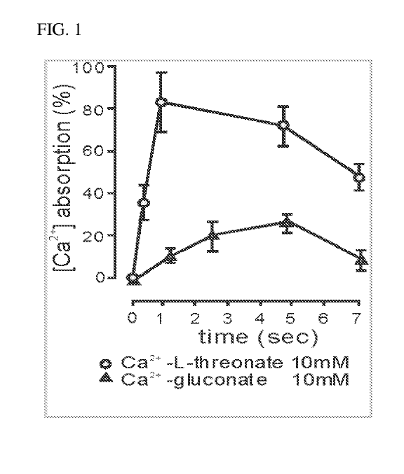

During scanning electron confocal microscopy, calcium L-threonate and calcium gluconate were applied separately, and then measured 1, 2.5, 5 and 7 sec time point calcium absorption (FIG. 1). The human bone marrow cells absorb approximately three times more calcium L-threonate than calcium gluconate at every time point tested, indicating that calcium L-threonate is absorbed more quickly by human cells than calcium gluconate (FIG. 1). Calcium L-threonate transports Ca²⁺ into blood via the intestine and deposits Ca²⁺ to the target organs, such as neurons, more efficiently.

In humans, depression is a long lasting and even life-threatening disorder with symptoms that include deficits of cognitive, psychomotor, and emotional processes. For example, the emotional effects of depression include, e.g., thoughts of profound sadness, emptiness, and hopelessness, and/or loss of interest or pleasure in normal activities. About one in six people in the U.S may succumb to depression at some point during their life spans (Kessler, R. (2005) “Lifetime prevalence and age-of-onset distributions of DSM-IV disorders in the National Comorbidity Survey Replication.” Arch Gen Psychiatry 62: 593-602). To test the antidepressant effects of calcium L-threonate, the emotional symptoms of depression in humans were reproduced in a mouse model using the tail suspension test (TST) and the forced swim test (FST).

TST is a widely used model for assessing and screening despair-based depressive behavior in mice (O'Leary and Cryan (2009) “The Tail-Suspension Test: A Model for Characterizing Antidepressant Activity in Mice.” Neuromethods 42: 119-137) The test is based on the observation that mice subjected to the short-term, inescapable stress of being suspended by their tails from a horizontal bar will eventually develop immobile postures after exhibiting escape-directed behaviors. Immobility is typically defined as the absence of initiated movements and includes passive swaying. Longer periods of immobility are associated with higher depressive scores. If an antidepressant treatment is given to the mice prior to the test, the mice will engage in escape-directed behaviors for longer periods of time than control mice given a placebo, e.g., saline. The duration of immobility is the main parameter by which the effectiveness of an antidepressant treatment is measured.

The FST, or Porsolt Test, is another widely used behavioral test for assessing depression in rodents and testing the efficiency of anti-depressants drugs and treatments (Porsolt et al. (1977) “Behavioral despair in mice: a primary screening test for antidepressants.” Arch Int Pharmacodyn Ther 229: 327-336; Petit-Demouliere et al. (2005) “Forced swimming test: a review of antidepressant activity.” Psychopharmacology (Berl) 177: 245-255). In an FST, each mouse is placed in a separate acrylic glass cylinder that is filled with water (23-25° C.) to a level high enough to prevent the mouse from touching the bottom of the cylinder with his paws or tail, yet low enough to prevent the mouse from escaping through the top opening of the cylinder. The mice are thus forced to swim in the cylinders. Mice that do not exhibit the behavioral symptoms of depression will try to swim out of the water-filled cylinders, even when they are unable to escape. In contrast, depressed mice will stop trying to escape and passively float in the cylinders, thus exhibiting behavioral despair. Mice given antidepressants have been observed to actively perform escape-directed behaviors for a longer duration than mice given, e.g., control saline treatment. FST is the most widely used as a screen for acute antidepressants, and the duration of immobility is the main parameter by which the effectiveness of an antidepressant treatment is measured.

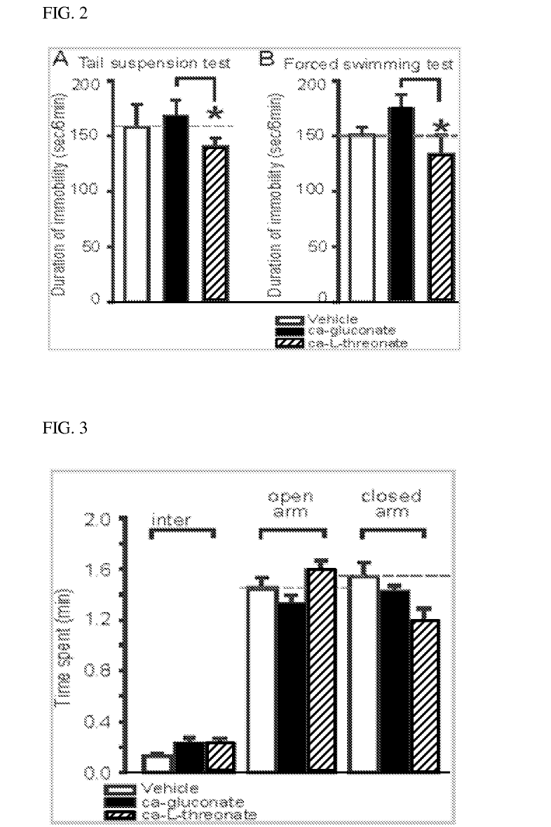

The tests described below were performed on three groups of 12 six-month-old male C57BL/6J mice. Calcium L-threonate was orally administered to the first group of mice, i.e., the test group, at a dose of 30 mg/kg/day of for 3 months (90 days). An equivalent dose of calcium gluconate was orally administered to a second group of mice for the same duration. Vehicle (saline) was administered to the third group of mice as a control. The animals were then subject to TST and FST. Mice given calcium L-threonate for 3 months were observed to exhibit a significantly decreased duration of immobility in both TST (FIG. 2A) and FST (FIG. 2B) tests compared to mice that were given calcium gluconate or vehicle (*, p<0.05; one way repeated measurement ANOVA). In other words, mice treated with calcium L-threonate prior to TST and FST exhibited increased escape-directed behaviors and, therefore, decreased symptoms of behavioral despair.

The mice's anxiety levels were assessed via elevated plus maze (EPM) test. EPM is a behavioral test that is commonly used to assess anxiety by measuring a mouse's locomotor activity and willingness to explore. When anxious, mice naturally prefer enclosed dark spaces to opened brightly lit spaces. In the EPM test, individual mouse from the test and control groups was placed on a plus-shaped apparatus that was raised from the floor and had two open and two enclosed arms. Within the context of EPM, anxious behavior was measured by the degree to which a mouse avoided the unenclosed arms of the maze. The number of entries and time spent in each arm was recorded and valid results were obtained in a single 5-minute testing session. The tests described below were performed on three groups of 12 six-month-old male C57BL/6J mice. Calcium L-threonate was orally administered to the first group of mice, i.e., the test group, at a dose of 30 mg/kg/day of for 3 months (90 days). An equivalent dose of calcium gluconate was orally administered to a second group of mice for the same duration. Vehicle (saline) was administered to the third group of mice as a control. As shown in FIG. 3, the mice given calcium L-threonate explored the open arms, whereas the mice given calcium gluconate or vehicle avoided the open arms and restricted their movements to the closed arms (p<0.05 calcium L-threonate vs. calcium gluconate; two way repeated measurement ANOVA). Taken together, these results indicate that calcium L-threonate can prevent or improve the depression-like symptoms.

What is claimed is:

1. A method for improving or preventing depression-like symptoms comprising administering an effective amount of calcium L-threonate to an individual.

2. The method of claim 1, wherein the calcium L-threonate is administered to the individual at a dosage of about 500 mg/day to about 1500 mg/day, and the individual is a human.

3. The method of claim 2, wherein the calcium L-threonate is administered to the individual at a dosage of about 600 mg/day to about 1200 mg/day.

4. The method of claim 1, wherein the calcium L-threonate is administered to the individual at a dosage of about 8.57 mg/kg/day to about 21.43 mg/kg/day.

5. The method of claim 1, wherein the calcium L-threonate is administered for at least about 3 month, at least about 6 months, or at least about 9 months.

6. The method of claim 1, wherein the calcium L-threonate is administered orally.

7. The method of claim 1, wherein the calcium L-threonate is provided in a nutritional supplement.

8. The method of claim 1, wherein the calcium L-threonate is provided in a composition comprising calcium L-threonate and a pharmaceutically acceptable carrier.

9. The method of claim 1, wherein the individual is an adult.

10. The method of claim 1, wherein the individual is a child.

11. The method of claim 1, wherein the individual has been diagnosed with depression.

12. The method of claim 1, wherein the individual has been selected for administration based on having depression-like symptoms.

13. The method of claim 1, wherein the individual has chronic stress.

14. The method of claim 1, wherein the administration of calcium L-threonate improves one or more of the depression-like symptoms in the individual.

15. The method of claim 14, wherein the symptom improved is a despair-based symptom.
